SEQUENCE LISTING (2 sequences)

SEQ ID NO: 1 - DNA, 20 bp, synthetic construct
  source 1..20: mol_type = other DNA
gcttatcggttcccaggaga

SEQ ID NO: 2 - DNA, 20 bp, synthetic construct
  source 1..20: mol_type = other DNA
cagggaccgagatcacatca

(19) United States
(12) Patent Application Publication
(10) Pub. No.: US 2025/0346964 A1
(43) Pub. Date: Nov. 13, 2025

(54) PRIMER FOR AMPLIFYING A MOLECULAR MARKER OF POWDERY MILDEW RESISTANCE GENE PMDR754 OF TRITICUM DICOCCON SCHRANK AND APPLICATION THEREOF

(71) Applicants: Crop Research Institute, Shandong Academy of Agricultural Sciences, Jinan (CN); Yantai University, Yantai (CN); National Center of Technology Innovation for Comprehensive Utilization of Saline-Alkali Land, Dongying (CN)

(72) Inventors: Cheng LIU, Jinan (CN); Pengtao MA, Yantai (CN); Yuli JIN, Yantai (CN); Xiaolu WANG, Jinan (CN); Kai WANG, Jinan (CN); Wenjing XU, Jinan (CN); Shengnan ZHAI, Jinan (CN); Guang QI, Jinan (CN); Jun GUO, Jinan (CN); Aifeng LIU, Jinan (CN)

(21) Appl. No.: 19/014,316

(22) Filed: Jan. 9, 2025

(30) Foreign Application Priority Data
May 8, 2024 (CN) 2024105613369

(51) Int. Cl. C12Q 1/6895; C12Q 1/686

(52) U.S. Cl. CPC C12Q 1/6895; C12Q 1/686; C12Q 2600/13

(57) ABSTRACT

The disclosure discloses a primer for amplifying a molecular marker of powdery mildew resistance gene PmDR754 of Triticum dicoccon Schrank, where the molecular marker primer includes a forward primer HENU765-F and a reverse primer HENU765-R; the nucleotide sequence of the forward primer HENU765-F is shown in SEQ ID NO:1, and the nucleotide sequence of the reverse primer HENU765-R is shown in SEQ ID NO:2. The molecular marker primer provided by the disclosure is applied to detection and identification of gene PmDR754 and auxiliary identification of a wheat powdery mildew resistance trait, and molecular marker-assisted selection breeding.

### TECHNICAL FIELD

The disclosure relates to the technical field of agricultural biological genes, and in particular to a primer for amplifying a molecular marker of powdery mildew resistance gene PmDR754 of Triticum dicoccon Schrank and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25004TLAN-USP1-SL.xml. The XML file is 2,926 bytes; is created on Jan. 9, 2025; and is being submitted electronically via patent center.

### BACKGROUND

Wheat is one of the important food crops, and its high and stable yield is particularly important for food security. Wheat powdery mildew, caused by the infection of Blumeria graminis f. sp. tritici, leads to a reduction in wheat production and poses a serious threat to the safe production of wheat. This disease is characterized by a variety of physiological races and rapid mutation, mainly infecting the leaves and stems of wheat, affecting photosynthesis of the leaves, and severely impacting the yield and quality of wheat. It is distributed in almost all major wheat-producing areas around the world. Measures to prevent and control wheat powdery mildew mainly include chemical control and the breeding and promotion of resistant varieties. Agricultural control, chemical control, and biological control cannot eradicate the occurrence of wheat powdery mildew from the source. Cultivating and exploring new resistant germplasm is currently the most effective, environmentally friendly, and economical method in the field.

Currently, in common wheat and its related species, more than 110 powdery mildew resistance genes and their alleles have been identified. Among them, powdery mildew resistance genes derived from common wheat can be directly introduced into target varieties and can be directly used for disease-resistant breeding, including Pm1a, Pm5e, Pm3b, Pm24a/b, Pm38/Lr34/Yr18/Sr57, Pm46/Yr46/Lr67/Sr55, etc.; Resistance genes from wheat's related species can be used to improve wheat resistance by introducing foreign fragments into common wheat through several generations of backcrossing, including Pm2, Pm8, Pm21, Pm58, etc. (Wang et al. Fighting wheat powdery mildew: from genes to fields. Theoretical and Applied Genetics, 2023, 136:196). Although several wheat resistance genes against powdery mildew have been identified to date, many of these genes have gradually lost their resistance or are difficult to utilize effectively. For instance, the Pm1a and Pm8 genes were once widely used in wheat production breeding due to their significant resistance to powdery mildew, but they have gradually lost their resistance in recent years due to the continuous mutation of the pathogen causing powdery mildew (Cowger et al. Wheat powdery mildew. Disease Resistance in Wheat, 2012, 84-119); The Pm12 gene, while highly resistant to powdery mildew, is associated with poor yield and quality traits, making it difficult to directly utilize in wheat breeding (Song et al. Molecular identification of Pm12-carrying introgression lines in wheat using genomic and EST-SRR markers. Euphytica, 2007, 158:95-102). Therefore, researchers in the industry need to continuously search for and map new powdery mildew resistance genes and apply them to wheat breeding for resistance to powdery mildew, in order to enrich the sources of wheat powdery mildew resistance.

Triticum dicoccon Schrank (2n=28, AABB) serves as a secondary gene pool for common wheat. It not only exhibits good resistance to various foliar diseases of wheat such as powdery mildew, leaf rust, and stripe rust, but also possesses characteristics that common wheat lacks, such as high protein content and rich in trace elements like zinc and selenium. Its superior genes can be directly transferred to common wheat through distant hybridization, making it a natural donor of exogenous excellent genes and an important resource for the genetic improvement of common wheat. Currently, four powdery mildew resistance genes have been reported in Triticum dicoccon Schrank, which are Pm5a located on chromosome 7BL, Pm49 on chromosome 2BS, and Pm4a and Pm50 on chromosome 2AL (Wang et al. Fighting wheat powdery mildew: from genes to fields. Theoretical and Applied Genetics, 2023, 136 (9): 196). Therefore, continuously exploring and researching Triticum dicoccon Schrank and its carried powdery mildew resistance genes, developing molecular markers, and applying them to marker-assisted selection breeding is of great significance for the development of wheat varieties resistant to powdery mildew, and thus effectively controlling the disease.

### SUMMARY

The disclosure aims to provide a primer for amplifying a molecular marker of powdery mildew resistance gene PmDR754 of Triticum dicoccon Schrank and an application thereof, the primer amplification molecular marker is used for positioning and detecting the wheat powdery mildew resistance gene PmDR754, the parent of the wheat powdery mildew resistance gene PmDR754 is purposefully selected in wheat breeding, and a guidance basis is provided for breeding new wheat varieties resistant to powdery mildew.

The disclosure is realized by the following method: a primer for amplifying a molecular marker of powdery mildew resistance gene PmDR754 of Triticum dicoccon Schrank is provided, where the molecular marker primer includes a forward primer HENU654-F and a reverse primer HENU654-R; the nucleotide sequence of the forward primer HENU654-F is shown in SEQ ID NO:1, and the nucleotide sequence of the reverse primer HENU654-R is shown in SEQ ID NO: 2.

The present disclosure provides an application of the primer for amplifying the molecular marker of powdery mildew resistance gene PmDR754 of Triticum dicoccon Schrank in detection and identification of gene PmDR754, auxiliary identification of a wheat powdery mildew resistance trait, and molecular marker-assisted selection breeding.

The disclosure further provides a method for detecting whether powdery mildew resistance gene PmDR754 of Triticum dicoccon Schrank is carried in a wheat sample, the method includes the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product; the molecular marker primer includes a forward primer HENU765-F and a reverse primer HENU765-R, the nucleotide sequence of the forward primer HENU765-F is shown as SEQ ID NO: 1, and the nucleotide sequence of the reverse primer HENU765-R is shown as SEQ ID NO:2; and
(3) performing electrophoresis and detection on the amplification product, where if a specific band of 131 bp is capable of being amplified, the wheat sample to be detected is indicated to carry the powdery mildew resistance gene PmDR754 of Triticum dicoccon Schrank, otherwise, the wheat sample to be detected is indicated not to carry the powdery mildew resistance gene PmDR754 of Triticum dicoccon Schrank.

A PCR amplification system in the method provided by the disclosure is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification in the method provided by the disclosure is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.

A procedure of the electrophoresis of the amplification product in the method provided by the disclosure is: performing electrophoresis on a non-denaturing polyacrylamide gel with the mass volume percentage concentration of 8%, after the amplification product is mixed with 2 μL of 6× sample loading buffer, taking 2 μL of the mixture for spot sampling, performing electrophoresis under constant pressure of 180 V for 2.5-3 hours, and performing photographing after silver nitrate staining.

According to the disclosure, a new dominant powdery mildew resistance gene PmDR754 is found in the Triticum dicoccon Schrank DR754, and a molecular marker HENU765 closely linked with the disease resistance gene is developed; through molecular marker linkage analysis and genetic segregation population detection, the molecular marker HENU765 of the powdery mildew resistance gene PmDR754 of wheat provided by the present disclosure is found to have a genetic distance of 0.027 cM from the gene PmDR754, is closely linked to the gene PmDR754, and is capable of being applied to the detection, identification, and auxiliary identification of wheat resistance to powdery mildew for the gene PmDR754, as well as marker-assisted selection breeding; it can also accurately detect the genetic mapping large population of the gene PmDR754, as well as the fine positioning and map-based cloning of the gene PmDR754. The primers for the molecular marker HENU765, which amplifies the wheat powdery mildew resistance gene PmDR754, are applied in the breeding of wheat resistant to powdery mildew. This not only allows for the rapid and precise selection of the powdery mildew resistance gene PmDR754, unaffected by environmental factors, with a clear target, but also saves production costs and greatly improves the efficiency and quality of selecting high-quality wheat varieties or lines resistant to powdery mildew.

### BRIEF DESCRIPTION OF THE DRAWING

FIG. 1 shows some results of detection of disease-resistant Triticum dicoccon Schrank DR754 and disease-sensitive Triticum turgidum Langdon (LDN) as well as segregation populations of hybrids thereof through a primer of a molecular marker HENU765.

In the figure, M: DL2000; 1: Triticum dicoccon Schrank DR754 (powdery mildew resistance variety carrying the gene PmDR754); 2: Triticum turgidum Langdon (powdery mildew susceptible variety); 3-17: F2:3 families formed by crossing the Triticum dicoccon Schrank DR754 with the Triticum turgidum Langdon, where 3-7: F2:3 family homozygous for resistance, 8-12: F2:3 family for resistance and susceptibility segregation, and 13-17: F2:3 family homozygous for susceptibility; and band indicated by a white arrow is specific band tracing the gene PmDR754.

### DETAILED DESCRIPTION OF THE EMBODIMENTS

The following embodiments are used for understanding the present disclosure better, but not intended to limit the present disclosure. Experimental methods in the following embodiments are conventional methods unless otherwise specified. All experimental materials, reagents, etc. used in the embodiments may be obtained from a commercial approach unless otherwise specified.

Embodiment 1 Development of molecular marker HENU765 primer of powdery mildew resistance gene PmDR754 of wheat

### Materials

Cultivated two-grain wheat DR754 and durum wheat Langdon (LDN) are resistant and susceptible to wheat powdery mildew, respectively. The cultivated two-grain wheat DR754 and durum wheat Langdon are crossed, and the Fi obtained is self-crossed, and the F2 population and corresponding F2:3 families are obtained Triticum dicoccon Schrank DR754 and Triticum turgidum Langdon (LDN) are resistant and susceptible to wheat powdery mildew respectively. F1 hybrids of the Triticum dicoccon Schrank DR754 and the Triticum turgidum Langdon are selfed to obtain a F2 population and corresponding F2:3 families.

### Genomic DNA Extraction of Wheat

A cetyltrimethylammonium bromide (CTAB) method for genomic DNA extraction of wheat includes the process as follows:
(1) Fresh wheat leaves are cut and ground with liquid nitrogen about 0.5 g of ground wheat leaves is put into a 2.0 mL centrifuge tube.
(2) 600 μL of CTAB extract solution is added and placed in a water bath at 65° C. for 40 minutes, where mixing up and down is performed once every 10 minutes.
(3) 600 μL of chloroform-isoamyl alcohol (24:1, v/v) is added and shaken gently on a shaking table for 15 minutes.
(4) Centrifugation is performed at 12000 rpm for 15 minutes, supernatant is taken to another 2 mL centrifuge tube, 3 times volume of precooled absolute ethanol is added, and precipitation is performed at a refrigerator at −20° C. for 60 minutes.
(5) Flocculent DNA precipitate is picked out and washed twice with 75% precooled ethanol.
(6) The DNA precipitate is picked out, placed in a 1.5 mL centrifuge tube, and air-dried indoors.
(7) 60 μL of TE buffer (100 mM Tris-Hcl, 10 mM EDTA, pH=8.0) is added to dissolve the DNA precipitate to prepare a DNA storage solution.
(8) The DNA storage solution is diluted to 20-30 ng/μL with ultrapure water as a working solution for later use.

### 3. Identification and Genetic Analysis of Powdery Mildew Resistance in Wheat Seedling Stage

Identification of wheat powdery mildew resistance at seedling stage is completed in artificial climate box. The resistance parent Triticum dicoccon Schrank DR754, the susceptible parent Triticum turgidum Langdon, the hybrid F₁, the F₂ population and the F_{2:3} families are planted in a 128-hole tray (2×2 cm), 5 seeds are sown in each hole, 20 seeds of each of the patents and F₁ are identified, 20 seeds of each F_{2:3} family are sown, and a susceptible control Mingxian 169 is sown randomly and labeled. After sowing, growth conditions are controlled as a 14 hours light/10 hours darkness cycle, a temperature of 20° C., and relative humidity of 30-40%. When the seedlings grow to a one leaf stage, and are inoculated with a wheat powdery mildew strain E09. The conditions for the first 24 hours after inoculation are controlled as follows: darkness, a temperature of 20° C., and relative humidity of 100%. Then, the conditions are controlled as follows: a 14 hours light/10 hours darkness cycle, a temperature of 18-22° C., and relative humidity of 100%. When the first leaf of the susceptible control Mingxian 169 is fully infected, phenotypes are recorded according to the standards of grades 0-4, where grades 0-2 are regarded as resistance grades, and grades 3 and 4 are regarded as susceptible grades.

The segregation population is constructed by using the disease-resistant parent DR754 and the susceptible parent Triticum turgidum Langdon. Inoculation and identification with the powdery mildew strain E09 shows that the F₁ hybrid generation exhibits resistance to the disease (IT: 0), and out of 229 individuals in the F₂ generation, 174 are resistant (IT: 0 or 0;), while 55 are susceptible (IT: 3 or 4). The x² test confirms that the segregation ratio is consistent with a 3:1 ratio (x²=0.071, P=0.78). Among the 167 resistant F_{2:3} families, 44 F_{2:3} families show homozygous resistance, and 123 F_{2:3} families exhibit a segregation of resistance and susceptibility. Additionally, 46 susceptible F_{2:3} families derived from F₂ individuals all show susceptibility, and their resistance segregation ratio conforms to a 1:2:1 ratio (x²=5.15, P=0.076). Combining the identification results from the F₁, F₂, and F_{2:3} generations, the resistance of the resistant parent, Triticum dicoccon Schrank DR754, to the powdery mildew strain E09 is controlled by a single dominant gene, which is named gene PmDR754.

### 4. Development of Markers Closely Linked to Gene PmDR754

Using the reported SSR primers, a wide screening of the resistant parent, Triticum dicoccon Schrank DR754, and the susceptible parent, Triticum turgidum Langdon, as well as the resistant and susceptible pools, has been conducted. The results reveal that the SSR marker Xbarc134, located on chromosome 6BL, exhibits polymorphism among the resistant and susceptible parents and between the resistant and susceptible pools. Using the SSR marker barc 134, genotyping is performed on 10 homozygous resistant and 10 homozygous susceptible plants from different families, and it is found that barc134 is linked to the powdery mildew resistance gene PmDR754. Based on the sequence information near Xbarc 134 from the Chinese Spring wheat reference genome, primers for Simple Sequence Repeat (SSR) markers are designed using the Primer5.0 software to genotype the DR754/LDN F_{2:3} families. This process leads to the identification of the SSR marker HENU765, which is closely linked to the PmDR754 gene, with a genetic distance of only 0.027 cM.

A primer of the molecular marker HENU765 includes a forward primer and a reverse primer:

a nucleotide sequence of the forward primer
HENU765-F:
(SEQ ID NO: 1)
5′-GCTTATCGGTTCCCAGGAGA-3′;
a nucleotide sequence of the reverse primer
HENU765-R: is
(SEQ ID NO: 2)
5′-CAGGGACCGAGATCACATCA-3′.

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoresis of an amplification product is: electrophoresis is performed on a non-denaturing polyacrylamide gel with the mass volume percentage concentration of 8%, after the amplification product is mixed with 2 μL of 6× sample loading buffer, 2 L of the mixture is taken for spot sampling, electrophoresis is performed under constant pressure of 180 V for 2.5-3 hours, and photographing is performed after silver nitrate staining.

If a specific band of 131 bp is simultaneously amplified, and wheat to be detected is indicated to carry the wheat powdery mildew resistance gene PmDR754; and if a specific band of 131 bp fails to be simultaneously amplified, and the wheat to be detected is indicated not to carry the wheat powdery mildew resistance gene PmDR754.

Embodiment 2 Application of molecular marker HENU765 of wheat powdery mildew resistance gene PmDR754

Using the primers of the molecular marker HENU765 obtained in Embodiment 1, the resistant parent Triticum dicoccon Schrank DR754, the susceptible parent Triticum turgidum Langdon (LDN), and the materials in the F_{2:3} families formed by their cross are detected. The extraction method is the same as in Embodiment 1.

Samples to be detected: the resistant parent Triticum dicoccon Schrank DR754, the susceptible parent Triticum turgidum Langdon (LDN), and the F_{2:3} families formed by their cross.

Genomic DNAs of the above materials are extracted as a template of PCR amplification, and the amplification is performed through the primer of the molecular marker HENU654 developed by the present disclosure:

nucleotide sequence of forward primer:
HENU765-F:
(SEQ ID NO: 1)
5′-GCTTATCGGTTCCCAGGAGA-3′;
nucleotide sequence of reverse primer:
HENU765-R:
(SEQ ID NO: 2)
5′-CAGGGACCGAGATCACATCA-3′.

A PCR amplification system is 10 μL, including: 1.0 μL of wheat genomic DNAs of 50 ng/μL, 5 L of a PCR Master Mix, 0.4 μL of a forward primer of 5 μM, 0.4 μL of a reverse primer of 5 M, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoretic segregation of an amplification product is: electrophoresis is performed on a non-denaturing polyacrylamide gel with the mass volume percentage concentration of 8%, after the amplification product is mixed with 2 μL of 6× sample loading buffer, 2 μL of the mixture is taken for spot sampling, electrophoresis is performed under constant pressure of 180 V for 2.5-3 hours, and photographing is performed after silver nitrate staining.

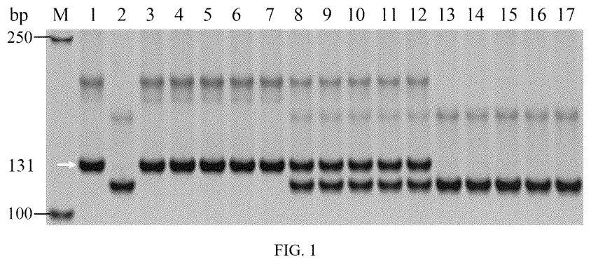

The results of molecular marker detection are shown in FIG. 1. FIG. 1 shows some amplification results of detection of disease-resistant Triticum dicoccon Schrank DR754 and disease-sensitive Triticum turgidum Langdon (LDN) as well as segregation populations of hybrids thereof by using primers of molecular marker HENU765. In the figure, M: DL2000; 1: Triticum dicoccon Schrank DR754 (powdery mildew resistance variety carrying the gene PmDR754); 2: Triticum turgidum Langdon (powdery mildew susceptible variety); 3-17: F_{2:3} families formed by crossing the Triticum dicoccon Schrank DR754 with the Triticum turgidum Langdon, where 3-7: F_{2:3} family homozygous for resistance, 8-12: F_{2:3} family for resistance and susceptibility segregation, and 13-17: F_{2:3} family homozygous for susceptibility; and band indicated by a white arrow is specific band tracing the gene PmDR754. The amplification results show that the specific band of 131 bp of the marker HENU765 is amplified in the disease-resistant parent Triticum dicoccon Schrank DR754 and the disease-resistant families, but not in the susceptible parent Triticum turgidum Langdon and the susceptible families.

According to the test results, the gene PmDR754 originated from the Triticum dicoccon Schrank DR765, and mapping and map-based cloning of the gene has not been reported yet. The molecular marker HENU765 and the primer provided by the present disclosure are used for detecting a large genetic mapping population, which contributes to fine mapping and map-based cloning of the gene PmDR754, and has very important significance for efficient transformation of the gene PmDR754 and in-depth analysis of a disease resistance mechanism.

The above embodiments are optimized implementation plans of the present disclosure, and are only used for illustrating the present disclosure, not limiting the present disclosure. All modifications or equivalent substitutions made by those skilled in the art without departing from the purpose and principle of the implementation plans of the present disclosure fall within the scope claimed to be protected by the present disclosure.

What is claimed is:

1. A primer for amplifying a molecular marker of powdery mildew resistance gene PmDR754 of Triticum dicoccon Schrank, wherein the molecular marker primer comprises a forward primer HENU765-F and a reverse primer HENU765-R, wherein the nucleotide sequence of the forward primer HENU765-F is shown as SEQ ID NO:1, and the nucleotide sequence of the reverse primer HENU765-R is shown as SEQ ID NO:2.

2. The application of the primer for amplifying the molecular marker of powdery mildew resistance gene PmDR754 of Triticum dicoccon Schrank according to claim 1 in detection and identification of gene PmDR754, auxiliary identification of a wheat powdery mildew resistance trait, and molecular marker-assisted selection breeding.

3. A method for detecting whether powdery mildew resistance gene PmDR754 of Triticum dicoccon Schrank is carried in a wheat sample, comprising the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product; the molecular marker primer comprises a forward primer HENU765-F and a reverse primer HENU765-R, the nucleotide sequence of the forward primer HENU765-F is shown as SEQ ID NO: 1, and the nucleotide sequence of the reverse primer HENU765-R is shown as SEQ ID NO: 2; and
(3) performing electrophoresis and detection on the amplification product, where if a 131 bp specific band is capable of being simultaneously amplified, the wheat sample to be detected is indicated to carry the powdery mildew resistance gene PmDR754 of Triticum dicoccon Schrank, otherwise, the wheat sample to be detected is indicated not to carry the powdery mildew resistance gene PmDR754 of Triticum dicoccon Schrank.

4. The method according to claim 3, wherein a PCR amplification system in step (2) is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

5. The method according to claim 3, wherein the procedure of the PCR amplification in step (2) is: performing predenaturation for 3 minutes at 94° C., performing denaturation for 15 seconds at 94° C. performing annealing for 20 seconds at 55° C. performing extension for 40 seconds, and performing 30 cycles; performing extension for 10 minutes at 72° C.; and performing preservation at 4° C.
